(19) United States
(12) Patent Application Publication
(10) Pub. No.: US 2006/0142684 A1
(43) Pub. Date: Jun. 29, 2006

(54) Oxygen releasing material

(75) Inventor: Edward Shanbrom, Santa Ana, CA (US)

Correspondence Address: LINER YANKELEVITZ SUNSHINE AND REGENSTREIF, 1100 GLENDON AVENUE, 14TH FLOOR, LOS ANGELES, CA, 90024, US

(73) Assignee: Shanbrom Technologies, LLC

(21) Appl. No.: 10/412,003

(22) Filed: Apr. 11, 2003

(51) Int. Cl. A61F 15/00; A61F 13/00

(52) U.S. Cl. 602/41; 602/43; 602/48

(57) ABSTRACT

An oxygen releasing bandage or dressing is based on a stable complex between polyvinyl acetal and hydrogen peroxide. Medical grade polyvinyl acetal sponge is treated with hydrogen peroxide, thereby forming a complex between the acetal plastic and the hydrogen peroxide. The dressing material is then affixed to a wound preferably with an oxygen impermeable covering. Fluids from the wound are drawn into the sponge where catalase and other enzymes in the fluids breakdown the hydrogen peroxide to release oxygen. If treatment of relatively dry wounds is desired, catalyst solutions can be introduced into the bandage to stimulate production of oxygen. The bandages are stable for a prolonged period of time.

### BACKGROUND OF THE INVENTION

1. Area of the Art

The present invention is in the area of compositions useful for medical treatments and more particularly for oxygen releasing compositions useful for wound treatment.

2. Description of the Prior Art

The recognition of the importance of sterility in wound protection and healing is less than two hundred years old. Not that long ago signs of inflammation and discharge were believed to be a prerequisite to effective wound healing. Today even children understand the importance of cleanliness in the treatment of wounds, and the sterility of bandages and wound dressings goes without question.

Yet, successful treatment of wounds due either to trauma, disease or medical operations often requires more than just an initially sterile dressing. Wound dressings are expected to protect the wound from contact or contamination and generally to absorb fluids exuded by the wound. This results in the dressing or bandage becoming saturated with fluids that are ideal breeding grounds for bacteria. Thus, the initially sterile bandage may actually become a source of infection. This has led to a long search for a germicidal bandage or wound dressing that inhibits bacterial multiplication within the dressing and perhaps even within the wound.

A large number of disinfectant or antibiotic materials have been added to bandage materials. Most simple disinfectant molecules have been employed. For example, iodine has long been a favored disinfectant for bandages. A number of disinfectant dressings based on povidone iodine (iodine bound to polyvinyl pyrrolidone) have been developed. Other iodine-binding materials have also proved useful. Attention is drawn to U.S. Pat. No. 5,071,648 to Rosenblatt, which describes an iodine releasing polymeric material.

Another disinfecting material is hydrogen peroxide which is highly germicidal in and of itself being an extremely strong oxidizer, but which also generates oxygen when decomposed by catalase, an enzyme found in most of the tissues exposed in a wound. The production of oxygen mediated by catalase is the primary reason that hydrogen peroxide foams when applied to may wounds. Hydrogen peroxide has not been used effectively in bandages although U.S. Pat. No. 5,674,436 to Breitenbach et al. (as well as the references cited therein) describes the production of stable complexes between vinyl polymers and hydrogen peroxide. These polymers constitute an oxygen producing powder that is useful for wound treatment.

The present inventor has also been interested in the problem of producing disinfecting materials and developed a variety of polymeric materials to which are bound disinfectant organic dyes. See for example, U.S. Pat. No. 5,811,471 to Shanbrom. Plastic foam made according to that patent has proved to be unexpectedly effective in the treatment of recalcitrant wounds such as bedsores and diabetic ulcers. As expected from the patent, the disinfectant dyes prevent growth of a variety of bacterial species within the bandage—thus preventing bandage-caused infection. What is more surprising given that the amount of disinfectant dye released from the bandage is extremely low, is the finding that over a period of time the material increases healing apparently by inhibiting bacterial growth within the wound.

However, there remains a variety of wounds that fail to heal optimally even when kept essentially sterile. In some cases it has been found that treatment of the wound with oxygen significantly improves healing. One approach has been so called hyperbaric treatment where the entire patient is placed into a chamber having an elevated partial pressure of oxygen. Although such an approach may succeed, there are often toxic systemic effects from excess oxygen.

A second approach has been to produce bandages that expose only the wound area to elevated concentrations of oxygen. U.S. Pat. No. 4,576,817 to Montgomery et al. describes a bandage that uses enzymes (such as glucose oxidase) to produce oxygen in a bandage or wound dressing. A drawback to this approach has been the lack of stability of the enzymes as well as the potential for the enzymes to produce allergic responses in the user. U.S. Pat. No. 5,855,570 to Scherson et al. discloses a rather complex bandage design that incorporates a fairly complex and expensive electronic oxygen-generating device. U.S. Pat. No. 6,000,403 describes a dome-shaped bandage that contains an oxygen gas reservoir.

Clearly there is a need for a simple and inexpensive oxygen-generating bandage for treatment of those conditions that benefit from elevated oxygen concentrations.

### SUMMARY OF THE INVENTION

An oxygen releasing bandage or dressing is formed from a polyvinyl acetal sponge containing a stable complex of hydrogen peroxide. The hydrogen peroxide complex can be created by soaking a polyvinyl acetal sponge in a hydrogen peroxide solution. More concentrated solutions provide a larger amount of the complex. Following complex formation the polyvinyl acetal sponge is dried at an elevated temperature. It is possible to form a hydrogen peroxide complex of 10% or more by weight of the sponge. Once formed the complex is stable for essentially an indefinite time.

All by itself the hydrogen peroxide containing sponge has significant disinfectant properties that enhance wound healing. When covered by an oxygen impermeable membrane, an effective oxygen releasing bandage or dressing is formed. When placed on a wound, wound fluids drawn into the sponge enzymatically breakdown the hydrogen peroxide to release oxygen. This is trapped at the wound site by the impermeable membrane and an oxygen rich atmosphere is created. Small pinholes or simple microscopic valves release the excess oxygen to the atmosphere and prevent an excess pressure of oxygen from developing.

One embodiment of the invention contains two layers of sponge separated by a liquid impermeable but oxygen permeable membrane. In such a situation, it is possible to introduce a catalytic solution into the upper layer to stimulate oxygen production while the lower layer in contact with the wound remains essentially dry. This arrangement makes oxygen production independent of enzymes released by the wound and prevents the added catalytic material from contacting the wound where it might be irritating or toxic.

### DESCRIPTION OF THE FIGURES

FIG. 1 shows a diagrammatic cross-section of a dressing or bandage of the present invention.

FIG. 2 shows a diagrammatic cross-section of a layered embodiment of the invention.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an oxygen releasing bandage based on polyvinyl acetal sponge.

The present invention is based on the observation that hydrogen peroxide forms stable complexes with organic polymers that are already used for bandages and wound treatment. Many surgical and other wound dressings and sponges are formed from polyvinyl acetal (also known as polyvinyl alcohol/acetal) (“PVAA”) foam. This material is highly absorbent and does not shed lint or other materials into a wound. As mentioned above it is known that iodine will complex with this polymer to form a useful disinfecting dressing. The present inventor has used PVAA to bind certain organic dyes to create a different disinfectant bandage or dressing material.

However, prior to the current invention it was not appreciated that hydrogen peroxide is capable of forming a stable complex with PVAA. The inventor discovered this property by soaking small PVAA sponges (originally designed for surgical dressings) in either dilute (about 3% wt./vol./) or concentrated (35% wt/vol.) forms. After the sponge is allowed to dry thoroughly, the presence of active hydrogen peroxide can be demonstrated. One means of demonstrating the presence of active hydrogen peroxide is treat the sponge with a few drops of 1-% sodium iodide (wt/vol.). Sodium iodide is an essentially colorless solution. When iodide is contacted by hydrogen peroxide, the iodide is oxidized to iodine, which appears as a brownish color on the PVAA sponge. As increasing amounts of iodine are generated, the iodine forms a blue-black complex with the PVAA (not unlike the well-known blue starch-iodine interaction). Therefore, the presence of hydrogen peroxide is easily determined by the formation of a blue-black color upon application of an iodide containing solution.

The hydrogen peroxide-PVAA complex is prepared by soaking a suitable PVAA sponge in a hydrogen peroxide solution. Generally soaking lasted at least about one hour, but the inventor has not determined the optimal soaking time. The hydrogen peroxide used is in an aqueous or alcoholic solution. Since the prior art discloses polyvinylpyrrolidone-hydrogen peroxide complexes formed using hydrogen peroxide in organic solvents, it seems likely that any solvent that does not damage the PVAA could be used. It appears that the interaction between PVAA and hydrogen peroxide is effective with low (e.g., 3%) as well as higher concentrations of hydrogen peroxide (e.g., 35%). However, more concentrated hydrogen peroxide solutions appear to result in formation of a larger weight of the hydrogen peroxide complex. Similarly, the complex formation occurs at room temperature as well as at elevated temperature. Elevated temperatures are generally those between room temperature and 100° C.

After soaking, the excess hydrogen peroxide solution is expressed from the PVAA sponge and the resulting sponge is allowed to dry either at room or elevated temperature. The hydrogen peroxide complex formed is stable (as determined by the iodide test) essentially indefinitely. By taking the sponge to dryness in an oven (120-140° C.) it is possible to determine that the treated PVAA sponge has gained weight (as compared to a control sponge treated with water instead of hydrogen peroxide). The sponge can gain more than 25% by weight hydrogen peroxide although 5-10% weight increases are more usual and are adequate for practice of the present invention.

The hydrogen peroxide complex is also stable if the sponges are not fully dried—that is, allowed to remain slightly moist. This may be an advantage for wound dressing purposes because such materials may remain softer and more flexible.

When PVAA-hydrogen peroxide is used as part of a wound dressing, there is a visible amount of foaming if the material comes into contact with the open wound. This is apparently due to the rapid release of oxygen caused by the enzymatic (primarily catalase and hemoglobin released from the wound) induced breakdown of hydrogen peroxide into oxygen. As contact with the wound continues obvious foaming decreases as the surface hydrogen peroxide complex becomes depleted. However, as the wound fluids diffuse more deeply into the material oxygen release continues for at least several hours (based on a PVAA thickness of 0.5 cm).

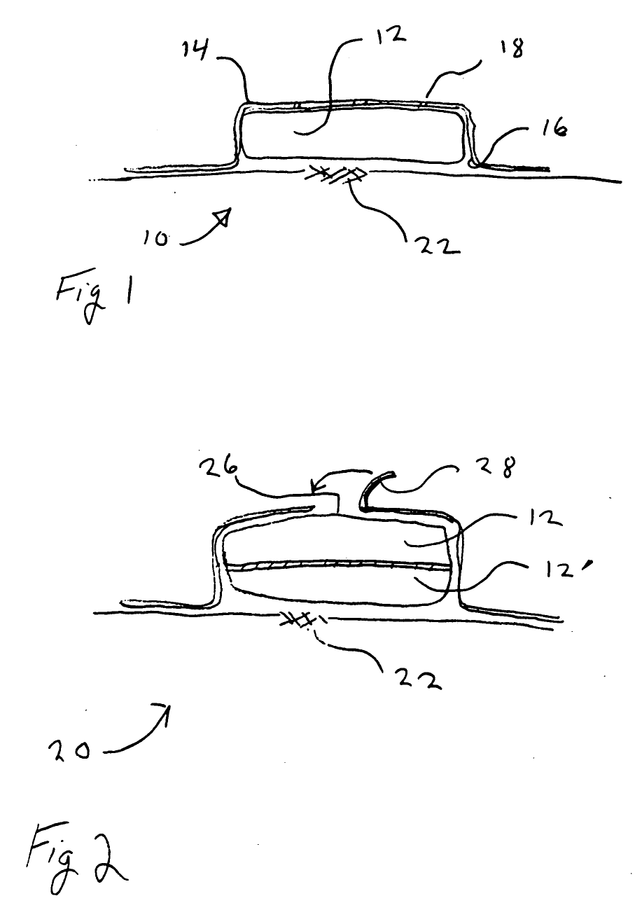

Simply taping a PVAA-hydrogen peroxide pad over a wound results in excellent healing. It is believed that the healing improvement is due to the immediate germicidal effect of the hydrogen peroxide and released oxygen. It is not clear that such a loosely taped PVAA-hydrogen peroxide pad achieves a significantly elevated concentration of oxygen around and in the wound. Therefore, while PVAA-hydrogen peroxide can be simply taped (or otherwise held) on a wound or be used as a pad component in a traditional adhesive bandage, a preferred configuration of a PVAA-hydrogen peroxide wound dressing is shown in FIG. 1.

In the dressing 10 of FIG. 1 a PVAA-hydrogen peroxide pad 12 is attached to the wound-facing surface of plastic member 14 having an adhesive coating 16. While the configuration appears similar to a traditional bandage, the plastic member 14 is specifically selected to have very low permeability to oxygen. There are a large number of low permeability plastic films known to those of skill in the art including, for example, polybutylene terephahalate, various metallized polymeric film, laminates of metal foil and plastic film, and compound films such as those composed of layers of ethylene-vinyl-acetate and ethylene-vinyl-alcohol. The addition of a plastic member 14 causes oxygen released by the hydrogen peroxide-PVAA complex in the pad 12 to become trapped and concentrated within the dressing and the wound. Small pinholes 18 can be made in the membrane 14 so that if oxygen evolution is particularly vigorous, the excess oxygen readily escapes to the atmosphere. Otherwise, oxygen pressure can cause partial lifting or release of the bandage 10. It is believed that an atmosphere of pure oxygen at or only slightly above atmospheric pressure is optimal. It is also possible to include elastic membranes closing the holes 18 or other simple valves to more specifically regulate the actual pressure.

When the inventive bandage 10 is applied to a wound 22, fluids from the wound are drawn into the pad 12 and enzymatic material in the wound fluids catalyze breakdown of the hydrogen peroxide and release of oxygen. Apart from oxygen release, the hydrogen peroxide complex is itself inherently antibacterial so that bacteria are not able to live within the bandage 10. This dual affect of preventing bacterial growth within the bandage 10 and providing enhanced oxygen levels within the wound prevent infection and speed wound healing.

In cases where the wound does not produce sufficient enzyme containing fluids to result in adequate oxygen production, it is possible to add liquid to the bandage 10 of the present invention prior to applying it to a wound. The added liquid should contain a small amount of a catalyst of hydrogen peroxide breakdown. Such catalysts are well known in the art and include enzymes such as hemoglobin and catalase as well as salts of transition metals such as ferric chloride. While it is possible to simply drop the catalytic solution onto the surface of the bandage, it may be advantageous to prevent contact of the solution with the wound. In such a case, the layered bandage of FIG. 2 may advantageously employed. The layered bandage 20 is similar in structure to the bandage 10 of FIG. 1; however, the PVAA pad is split into two layers 12 and 12′. An oxygen permeable but water impermeable membrane 24 separates the two layers. Such an oxygen permeable membrane 24 can readily be formed from expanded polytetrafluoroethylene film although films of other fluorocarbons as well as other materials are also useable. The catalytic solution is introduced into the upper layer 12 (this can be done either immediately before attaching the bandage 20 to the wound or immediately thereafter. The solution can be injected into the bandage using a needle that penetrates the plastic member 14. Alternatively, an opening 26 can be provided in the plastic member 14, which opening is then closed by an additional layer of plastic membrane 28 after introduction of the catalytic solution.

The catalytic solution causes breakdown of hydrogen peroxide and concomitant release of oxygen. The oxygen is trapped by the plastic membrane 14 and diffuses through the oxygen permeable membrane 24 and into the wound. At the same time, the oxygen permeable membrane 24 prevents the wound from coming into contact with the catalytic solution.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### REF LABELS

FIG. 1 shows a diagrammatic cross-section of a dressing or bandage of present invention.

FIG. 2 shows a diagrammatic cross-section of a layered embodiment of invention.

| | |
|---|---|
| 10 | whole dressing |
| 12 | pvaa pad |
| 14 | plastic member |
| 16 | adhesive |
| 18 | pin holes |
| 20 | layered |
| 22 | wound |
| 24 | oxygen permeable |
| 26 | opening |
| 28 | sealing layer |

I claim:

1. A bandage or dressing for providing oxygen to stimulate wound healing comprising a polyvinyl acetal sponge containing hydrogen peroxide-polyvinyl acetal complex.

2. The apparatus according to claim 1, wherein the sponge contains more than one percent by weight hydrogen peroxide.

3. The apparatus according to claim 1, further comprising an oxygen impermeable membrane covering the sponge.

4. The apparatus according to claim 3, further comprising means for preventing an excessive pressure of oxygen in the bandage.

5. The apparatus according to claim 3 wherein the sponge is divided into an upper layer in contact with the oxygen impermeable membrane and a lower layer with an oxygen permeable and liquid impermeable membrane therebetween.

6. The apparatus according to claim 5 equipped with means for introducing a liquid into the upper layer.

7. A dressing or bandage for providing oxygen to a wound comprising:
a polyvinyl acetal sponge containing polyvinyl acetal-hydrogen peroxide complex; and
an oxygen impermeable membrane to cover the sponge and retain oxygen.

8. The apparatus according to claim 7, wherein the sponge contains more than one percent by weight hydrogen peroxide.

9. The apparatus according to claim 7, further comprising means for preventing an excessive pressure of oxygen in the bandage.

10. The apparatus according to claim 7 wherein the sponge is divided into an upper layer in contact with the oxygen impermeable membrane and a lower layer with an oxygen permeable and liquid impermeable membrane therebetween.

11. The apparatus according to claim 10 equipped with means for introducing a liquid into the upper layer.

12. The apparatus according to claim 11, wherein the means for introducing a liquid comprises an opening through the plastic membrane and a flap of plastic membrane for closing the opening.

13. A method for producing an oxygen generating bandage material containing hydrogen peroxide-polyvinyl acetal complex comprising the steps of contacting polyvinyl acetal with hydrogen peroxide and removing unbound hydrogen peroxide thereby leaving polyvinyl acetal-hydrogen peroxide complex.
